(19) United States
(12) Patent Application Publication
(10) Pub. No.: US 2023/0298733 A1
(43) Pub. Date: Sep. 21, 2023

(54) Systems and Methods for Mental Health Improvement

(71) Applicant: North Shore Therapeutics LLC, Boylston, MA (US)

(72) Inventors: Josef M. Powers, Alexandria, VA (US); Sunny Tang, Kings Park, NY (US); Aaron Philip Brinen, Nashville, TN (US); Michael Birnbaum, Rhinebeck, NY (US)

(21) Appl. No.: 18/041,451

(22) PCT Filed: Aug. 12, 2021
(86) PCT No.: PCT/US2021/045686

§ 371 Date: Feb. 13, 2023

Related U.S. Application Data
(60) Provisional application No. 63/064,492, filed on Aug. 12, 2020.

(51) Int. Cl. G16H 20/70; A61B 5/16; G16H 50/20; G06T 19/00

(52) U.S. Cl. CPC G16H 20/70; A61B 5/165; A61B 5/162; G16H 50/20; G06T 19/006

(57) ABSTRACT

Systems and methods are provided for improvement of mental health of a subject that avoids adverse side effects and may be safely administered over long periods of time, such as to address chronic conditions. The systems and methods include use of immersive technology, such as augmented reality environments, which may be used as an effective treatment for negative symptoms of mental illness systems or to promote mental health.

### CROSS-REFERENCE TO RELATED APPLICATIONS

This application claims the benefit of U.S. Provisional Patent Application Ser. No. 63/064,492 filed on Aug. 12, 2020 and entitled “Systems and Methods for Mental Health Improvement,” which is incorporated herein by reference as if set forth in its entirety for all purposes.

### STATEMENT REGARDING FEDERALLY SPONSORED RESEARCH

N/A

### BACKGROUND

Mental illness may lead to positive and/or negative symptoms. Positive symptoms are typically characterized by the presence of problematic behavioral, emotional and cognitive attributes that signal underlying mental illness. For example, hallucinations and delusions are vivid and conspicuous positive symptoms of schizophrenia. Negative symptoms are typically characterized by deficits or absence of behavioral, emotional and cognitive attributes that are necessary for good mental health. Negative symptoms, for example, are a strong predictor of poor functional outcome in people with schizophrenia. Chronic disabilities, including social, educational and occupational dysfunction, unhealthy lifestyle choices (e.g. poor nutrition, lack of exercise, and substance abuse) result primarily from negative symptoms, leading to severe burdens on the afflicted, their families and society generally.

Conventionally, there are numerous psychopharmacological treatments for positive symptoms, but the efficacy of such drugs for negative symptoms is severely limited. Unpleasant and occasionally disabling side effects render currently available psychopharmacological treatments unsuitable for treatment of chronic conditions such as negative symptoms. Unfortunately, there are few, if any, effective interventions for either negative symptoms or functional outcomes, despite the identification of potential mechanisms.

For the foregoing reasons, there remains a need for effective treatment for negative symptoms that do not cause adverse side effects and can be safely administered over long periods of time to address chronic conditions.

### SUMMARY OF THE DISCLOSURE

The present disclosure addresses the aforementioned drawbacks by providing systems and methods for improvement of mental health that avoids adverse side effects and may be safely administered over long periods of time, such as to address chronic conditions. The systems and methods may be used as an effective treatment for negative symptoms of severe mental illness, including schizophrenia and the like, or for improvement of mental health in general.

In one configuration, a computer-implemented method is provided for improving mental health of a subject. The method includes obtaining, using a processor, a mental health treatment protocol based on an initial assessment of the subject. The method also includes generating, using the processor, an immersive experience for the subject to implement the mental health treatment protocol and to facilitate the subject to enter an adaptive mode. The method also includes generating, using the processor, a challenge activity for the subject in the immersive experience and assessing the subject's success with the challenge activity associated with the immersive experience. The method also includes updating, using the processor, the mental health treatment protocol based upon the success of the subject in the immersive experience.

In some configurations the immersive experience includes a virtual reality, an augmented reality, a synthetic environment, or a mixed reality. In an example, the immersive experience is an augmented reality that includes real artifacts overlaid with synthetic artifacts generated for the augmented reality.

In some configurations the challenge activity includes a puzzle that is configured to facilitate the subject to enter the adaptive mode. In some configurations, the challenge activity is configured to improve mental health of the subject.

In some configurations, assessing the subject's success with the challenge activity includes determining a duration of the challenge activity, a difficulty of completing the challenge activity, retained learning from the challenge activity, or subject fatigue. In some configurations, the treatment protocol includes a plurality of treatment episodes, each of which include a plurality of generated immersive experiences.

In some configurations, assessing the subject's success with the challenge activity is performed after each immersive experience, and subsequent immersive experiences are adjusted based upon a preceding assessment of the subject's success with the challenge activity. Assessing the subject's success with the challenge activity may include using a microphone, a video camera, and a biometric sensor to acquire data of the performance of the subject.

In some configurations, the initial assessment includes an assessment of avolition, asociality, or anhedonia in the subject. In some configurations, a mental health scoreboard is used and may be calibrated based on the initial assessment of the subject. The mental health scoreboard may include scoring the subject in areas of at least one of experience, belief system, or functional targets.

In one configuration, a non-transitory computer-readable medium is provided. The computer-readable medium includes stored instructions that, when executed by a computer, cause the computer to perform a method. The method includes obtaining, using a processor, a mental health treatment protocol based on an initial assessment of the subject. The method also includes generating, using the processor, an immersive experience for the subject to implement the mental health treatment protocol and to facilitate the subject to enter an adaptive mode. The method also includes generating, using the processor, a challenge activity for the subject in the immersive experience and assessing the subject's success with the challenge activity associated with the immersive experience. The method also includes updating, using the processor, the mental health treatment protocol based upon the success of the subject in the immersive experience.

The foregoing and other aspects and advantages of the present disclosure will appear from the following description. In the description, reference is made to the accompanying drawings that form a part hereof, and in which there is shown by way of illustration a preferred embodiment. This embodiment does not necessarily represent the full scope of the invention, however, and reference is therefore made to the claims and herein for interpreting the scope of the invention. Like reference numerals will be used to refer to like parts from Figure to Figure in the following description.

### BRIEF DESCRIPTION OF THE DRAWINGS

FIG. 1 is a flowchart of non-limiting example steps for a method of improving mental health for a subject.

FIG. 2 is a flowchart of non-limiting example steps for a method of sensory processing by a subject with a cognitive status in patient mode.

FIG. 3 is a flowchart of non-limiting example steps for a method of sensory processing by a subject with a cognitive status in adaptive mode.

FIG. 4 is a non-limiting example diagram of a treatment protocol in accordance with the present disclosure.

FIG. 5 is a diagram of a non-limiting configuration for a system for improving mental health in accordance with the present disclosure.

FIG. 6 is a diagram of a non-limiting example system capable of generating an immersive environment in accordance with the present disclosure.

FIG. 7 is a block diagram of a computer system capable of operating the systems and methods in accordance with the present disclosure

### DETAILED DESCRIPTION

Systems and methods are provided for improvement of mental health that avoid adverse side effects and may be safely administered over long periods of time, such as to address chronic conditions. In some configurations, the systems and methods may be used as an effective treatment for negative symptoms of mental illness. In a non-limiting example, systems and methods of the present disclosure may apply immersive technology to address mental illness or to promote mental health. The systems and methods may include the use of immersive experiences, such as virtual, augmented, or mixed reality with activities or challenges configured to facilitate an adaptive mode in a subject for treating a subject with a mental illness or mental health concerns. The active engagement of a subject with the immersive experiences and challenges may offer additional advantages over conventional treatment methods that rely upon passive talking sessions or drug therapy, such as with increased efficacy of treatment, reduced duration of treatment needed to achieve a goal for the subject, a reduced reluctance by the subject to engage with the treatment, increased access such as with a telehealth application, decreased expense, and the like.

For purposes of the present disclosure, a “mode” is a manner of thinking and acting that involves beliefs, attitudes, emotion, motivation and behavior. Non-limiting example modes include survival mode, vacation mode, defeatist mode, adaptive mode, and the like. “Survival mode” is the short-term, stressful, fear-based mode of thinking that hyper-focuses attention on a perceived threat when the fight-or-flight response is triggered. By contrast, “vacation mode” may be characterized by a relaxed involuntary attention whereby we are free to imagine, daydream or enjoy nature. “Defeatist mode” is a mental state dominated by frustration, regret, worry and fear. Defeatist mode is characterized by excessive regret over past setbacks and fear of what might go wrong in the future. The “adaptive mode”, which has been described as an “at-your-best” state of mind, is characterized by motivation (a willingness to engage in social activity), optimism and enjoyment. By contrast, “patient mode” is a mode where a subject is unwilling and/or unable to engage in learning or social activity, is pessimistic, lacks enjoyment, and the like. A “patient mode” may be considered as the complement of “adaptive mode” (e.g. anything other than “adaptive mode”).

A basic Recovery-Oriented Cognitive Therapy (CT-R) model may include the concept of recovery, the cognitive model, and the idea of modes. Conventional cognitive behavioral therapy (“CBT”) is an evidence-based talking therapy that concentrates on how an individual's thoughts, behaviors, and emotions are connected. CBT helps individuals become aware of their thoughts (ie interpretation of events) and behaviors, with a focus on exploring how these impact their emotions. CBT is appropriate for treatment of mild to moderate depression and anxiety, but not for treatment of more serious mental health issues. CBT may also be used as an adjunct to psychopharmacological medication. Psychosocial interventions were long overlooked as a treatment for psychosis. Cognitive Behavioral Therapy for psychosis (CBTp) is primarily focused on positive symptom reduction as a way to reduce distress associated with the positive symptoms of psychosis. It has emerged as an evidence-based intervention recommended as an adjunct to medication management.

In a recovery model for mental health, people with mental illness are recognized as being able to participate in the mainstream of society, based on evidence demonstrating that many people eventually recover from serious mental illness. The United States Surgeon General David Satcher's 1999 report on mental health states “All services . . . should be consumer oriented and focused on promoting recovery . . . [T]he goal of services must not be limited to symptom reduction but should strive for restoration of a meaningful and productive life.” Recovery-oriented clinical care may include identifying and building on people's strengths and the opportunities and resources that exist in their communities, and empowering people to play an active role in the management of their disease.

CT-R is a treatment approach that may be used to promote recovery in individuals with serious mental health conditions. CT-R recovery means patients connect, or reconnect, with other people and achieve the life they wish to live. Different from CBTp, CT-R focuses on activating adaptive modes of living, developing meaningful aspirations, and engaging in personally meaningful activities to bring about one's desired life. CT-R is particularly helpful for people who might otherwise not engage in treatment due to mistrust, chronic institutionalization, limited access to motivation, limited verbalization, or intensity of symptom presentation.

For a subject, functional targets may be quantified functional goals such as attending full-time school or full-time job or equivalent, close relationships with family or friends, physical fitness and healthy routines, prosocial activities like volunteer work, helping others, and the like. The subject, such as a patient, identifies important goals in the beginning of a method for improving mental health, and components of such goals can play into the real world suggestions along the way. The function tracker may monitor from 0 to 100% as the patient meets those functional goals.

In some configurations, CT-R may involve the activation of the adaptive mode in people who would otherwise be stuck in defeatist mode. By eliciting activity that generates positive feedback, CT-R may be used to awaken the dormant adaptive mode in a subject, gradually releasing the patient from their defeatist entrapment. Once in adaptive mode, patients are receptive to learning new, healthier belief systems. Over time, consistent CT-R therapy allows patients to unlearn the defeatist belief schema that holds them back from achieving their aspirations.

In some configurations, systems and methods of the present disclosure apply immersive technology and methods to liberate patients stuck in a defeatist mode by activating, energizing, developing, and strengthening their adaptive mode, thereby leading to functional recovery from mental illness.

Immersive technology may extend or augment a subject's perceived environment, and/or create a new perceived environment, by generating sensory input via a device or system of devices. Sensory input may include visual, auditory, haptic, olfactory, taste, or direct neural stimulation such as galvanic vestibular stimulation for immersive motion perception, and the like. Immersive technology may include interactive “360-degree” video, virtual reality through head mounted displays with motion and/or position sensors (“VR”), augmented reality where simulated artifacts are superimposed on the real environment (“AR”), and mixed reality which combines aspects of VR and AR.

An immersive experience is a continuously sustained interaction for the duration of treatment between a subject and a partially synthetic sensory environment. In a non-limiting example, an immersive experience may be sustained for a particular time interval, such as a subset of time of a treatment, and may engage the patient in a directed activity. A treatment episode may be a contiguous sequence of one or more immersive experiences and associated directed activities. Each such treatment episode may engage the patient in a directed activity, such as a challenge.

A challenge may include any activity configured to test or treat a psychological condition of a subject. In a non-limiting example, a challenge may include directing a person who is fearful of the outdoors to exit a confined synthetic environment into an outdoor synthetic environment, or for a person fearful of interacting with others to interact with another subject in the synthetic environment, and the like. In a non-limiting example, a challenge may include a puzzle, and the puzzle may be configured to facilitate the subject to enter the adaptive mode in order to be receptive to treatment. Success or failure with a challenge or activity may include determining how long it took a subject to complete the challenge, how difficult the challenge was for the subject, whether there is retained learning from the challenge, whether a subject was fatigued by the challenge, and the like.

In a non-limiting example, successful episodes may be acknowledged, the scoreboard updated, and the system may further assess whether to engage a patient in a new challenge/activity or to conclude the treatment session. Conclusion of a treatment session may be based upon expiration of time, patient fatigue, and the like. In non-limiting examples, failed episodes may be followed by coaching, adjustment of the scoreboard/protocol, an adaptive mode activating immersive experience, an assessment of whether to continue with another challenge or conclude the treatment session, and the like. A treatment protocol may include a sequence of treatment episodes.

In some configurations, the immersive experience or a treatment episode, or the treatment protocol may be adaptive. Adaptation may include adjustments made based upon an assessment of the patient or subject. Adjustments may include changing a length of time for a session, adjusted coaching, adjustment of the scoreboard/protocol, an adjustment to the mode activating immersive experience, changing to another challenge, or conclusion of the treatment session, and the like.

Monitoring of patient status, such as for an assessment, may be performed through observation by a clinician observer, automatically by sensors, self-monitoring by the patient, by the system or by a combination of two or more of these monitoring modalities. Monitoring may include measurements of interactions with the system, self-assessment, facial expression, heart rate, therapist assessment, and the like. In non-limiting examples, self-assessment may include answers to interactive questions posed through an interface for the subject, and the like.

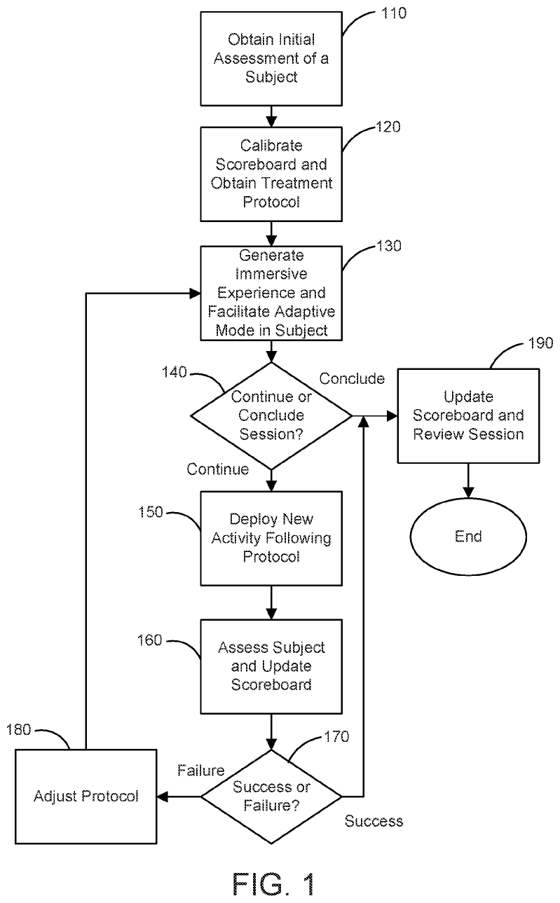

Referring to FIG. 1, a flowchart of non-limiting example steps for a method of improving mental health for a subject is shown. An initial assessment of a subject may be obtained at step 110 to determine a treatment protocol for the subject, or a treatment protocol may be obtained based upon a previous initial assessment. In non-limiting examples, an initial assessment or treatment protocol may be obtained from clinical notes for a subject, from an archive, a previous patient visit, or otherwise acquired from a medical record. In some configurations, an initial assessment may be performed at step 110. The initial assessment may quantify the subject's levels of avolition, asociality, anhedonia, and the like. The initial assessment may also characterize a subject's beliefs and identify functional targets. In some configurations, a scoreboard may be used to score the subject's mental health state by providing a numerical score to the subject's levels of avolition, asociality, anhedonia, or to quantify the level of phobia or stress a subject is suffering, and the like. A scoreboard may be calibrated for the subject at step 120 based upon the initial assessment, and a treatment protocol may be determined or generated based upon the initial assessment. The scoreboard may quantify a subject's experience, belief system, functional target progress, and the like. Calibration of the scoreboard may include establishing a baseline for the subject's experiences, belief system, functional target progress, and the like, prior to engaging in treatment. In non-limiting examples, the scoreboard may include scores for the presence or absence of a condition, such as a score between 0 and 1, or a scoreboard may include a degree for a condition such as with scores between 0-10, or between 0-100 or any other numerical range to quantify the subject's mental health state.

An immersive experience may be generated for the subject based upon the determined treatment protocol, and an adaptive mode in a subject may be facilitated or activated at step 130 by engaging the subject in an experience or challenge within the immersive experience. An immersive experience may include a synthetic, virtual, or augmented reality environment as described above with the experience or challenge configured for completion within the synthetic, virtual, or augmented reality environment. As described above, challenges may include puzzles or any activity configured to test or treat a psychological condition of a subject. In a non-limiting example, the immersive experience is a virtual reality generated by a computer system and the challenge is a puzzle designed to test the cognitive or psychological state of the subject.

An assessment of whether to continue or to conclude a treatment session or an experience may be performed at step 140 following the protocol. If the subject has successfully or satisfactorily completed the challenge, the assessment indicates to conclude the session, and the treatment session may be reviewed and the scoreboard updated with the conclusion of the session at step 190. If the assessment indicates to continue either due to an incomplete challenge, an unsuccessful challenge, or any other mode of failure, a new activity for the subject may be deployed at step 150 following the protocol. The subject may be assessed and the scoreboard updated at step 160 following the new activity. Success or failure of the new activity may be determined at step 170. A determination of failure at step 170 may result in adjustment of the protocol at step 180 before returning to step 130 to resume treatment. A determination of success at step 170 may result in acknowledgment of the success with either adjustment of the protocol or a conclusion of the session.

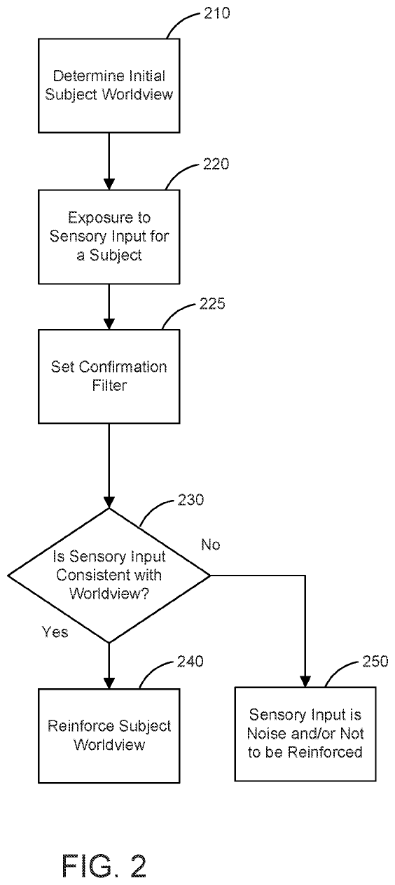

Referring to FIG. 2, a flowchart of non-limiting example steps for a method of sensory processing by a subject with a cognitive status in patient mode, is shown. In a patient mode, a subject may be predisposed to reject an input, or to reject a therapy, or to reject general learning. According to FIG. 2, a subject following a patient mode method may determine an initial subject worldview at step 210. A subject may be exposed to a sensory input at step 220. In a non-limiting example, an immersive experience may form the sensory input for step 220. A confirmation filter may be set to “on” at step 225, representing filtering by the subject's neural circuitry and indicating that the subject is disinclined to accept the sensory input as part of the subject's initial worldview. A determination of whether the sensory input is consistent with the initial worldview is performed at step 230. If the determination is consistent, then reinforcement of the subject's worldview is performed at step 240. If the determination is inconsistent, then the sensory input may be noise and/or reinforcement is not to be performed according to step 250.

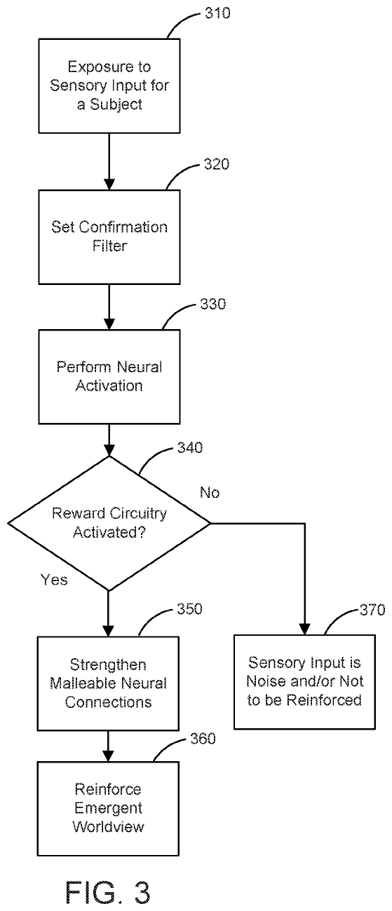

Referring to FIG. 3, a flowchart of non-limiting example steps for a method of sensory processing by a subject with a cognitive status in adaptive mode, is shown. In an adaptive mode, a subject may be predisposed to accept an input, to learn, or to accept a therapy as a treatment. According to FIG. 3, a subject may be exposed to a sensory input at step 310. In a non-limiting example, an immersive experience may form the sensory input for step 310. A confirmation filter may be set to “off” at step 320, representing filtering by the subject's neural circuitry is not taking place, and indicating that the subject is inclined to accept the sensory input as part of the subject's initial worldview. Neural activation of the subject may be performed at step 330. A determination of whether the reward circuitry of a subject is activated by the neural activation is performed at step 340. If the reward circuitry is activated, then malleable neural connections may be strengthened at step 350 and an emergent worldview of the subject may be reinforced at step 360. If the reward circuitry is not activated at step 340, then the sensory input may be noise and/or an emergent worldview is not to be reinforced according to step 250.

Referring again to FIG. 1, in some configurations, facilitating or activating an adaptive mode for a subject at step 130 may include switching a subject from a patient mode to an adaptive mode. This may be accomplished by using the systems and methods in accordance with the present disclosure, such as by engaging the subject in an immersive experience and using directed activities/challenges. In a non-limiting example, switching a subject from a patient mode to an adaptive mode may result in a subject progressing through a therapy to achieve favorable results.

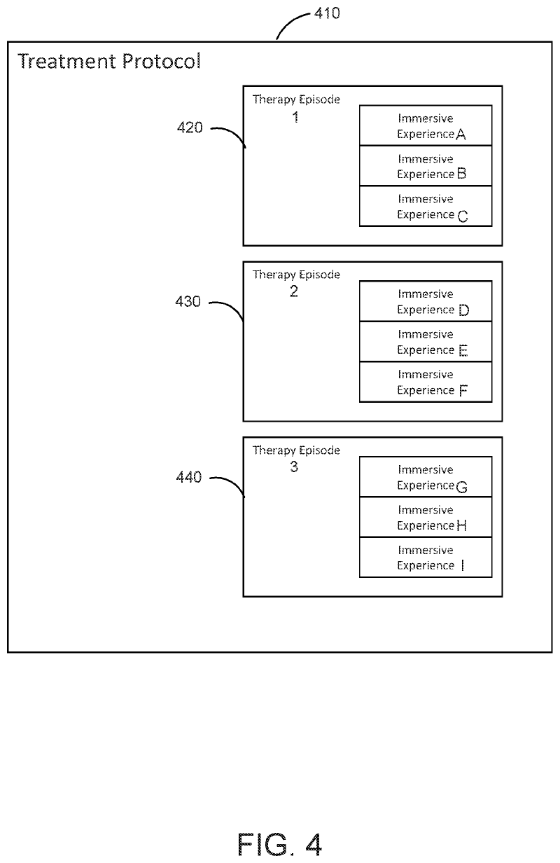

Referring to FIG. 4, a non-limiting example diagram of a treatment protocol 410 is shown. Treatment protocol 410 may include first therapy episode 420, which includes a number of immersive experiences, such as immersive experiences A, B, and C as shown. Treatment protocol 410 may also include second therapy episode 430, which includes a number of immersive experiences, such as immersive experiences D, E, and F as shown. Treatment protocol 410 may also include third therapy episode 440, which includes a number of immersive experiences, such as immersive experiences G, H, and I as shown. It is to be appreciated that these are non-limiting examples and that any number of therapy episodes may be used in a treatment protocol, and that any number of immersive experiences may be used in each therapy episode.

In some configurations, immersive experience applications can be used to generate and/or provide immersive experiences. Subject data can include various information regarding an individual including medical history, responses to previous treatment, personal preferences, and the like. Subject data may be acquired using any appropriate system, such as video, audio, text, prompts, 3D models, and the like, or other elements that can be provided as a part of an immersive experience.

In some configurations, treatment protocols may be generated with a treatment sequence for a plurality of sessions with a subject. Treatment sequences can be tailored to a subject based on numerous factors including medical history, motion sensitivity, progress within a treatment, progression of treatment sessions, and the like. Generating treatment sequences can include selecting media content, generating environments for a training session, and the like. Generated environments used in an immersive experience may include virtual and/or augmented reality. Augmented environments can include visual, auditory, olfactory, tactile elements, and the like.

In some configurations, a subject's responses to a treatment session may be recorded. Recordings can include application telemetry, changes in heart rate, signs of stress, facial expressions, and the like, that are detected from a subject. Detection may be performed by video analysis, audio analysis, sensors such as electrocardiograph recordings and the like, or other sensory inputs. In some configurations, the recordings from a subject may be used to direct prompts provided to the subject, to modify a treatment sequence, and/or to be provided to a clinician for professional for review. In a non-limiting example, the heartrate of a subject fearful of the outdoors may be monitored while the subject is directed to exit a virtual indoor space into a virtual outdoor space, and the change in the heartrate may be used to gauge the success or progress of the patient. A highly increased heartrate would indicate less success or less progress and would be scored lower, such that the subject may be exposed to additional such events until a normal or a reduced level of increase in the heartrate is achieved.

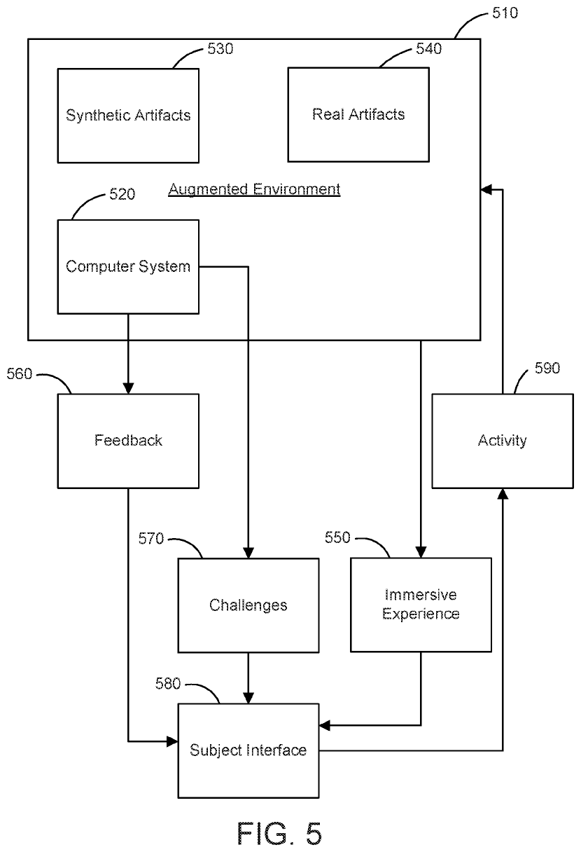

Referring to FIG. 5, a diagram of a non-limiting system configuration for a system for improving mental health is shown. The system includes an augmented environment 510. The augmented environment 510 may be generated by computer system 520, such as a tablet or smartphone in non-limiting examples. Synthetic artifacts 530 may be generated by the computer system 520, and real artifacts 540 included from the real environment. An immersive experience 550 may be a subject's perception of the augmented environment 510, where the augmented environment 510 may use the combination of synthetic artifacts 530, real artifacts 540, and a treatment protocol to create the immersive experience 550. A subject may be provided with feedback 560 and may be presented with challenges 570 through a subject interface 580. Subject interface 580 may provide a subject with the ability to accept challenges 570 by engaging in an activity 590 that may interact with the augmented environment 510.

In some embodiments, any suitable computer readable media can be used for storing instructions for performing the functions and/or processes described herein. For example, in some embodiments, computer readable media can be transitory or non-transitory. For example, non-transitory computer readable media can include media such as magnetic media (e.g., hard disks, floppy disks), optical media (e.g., compact discs, digital video discs, Blu-ray discs), semiconductor media (e.g., random access memory (“RAM”), flash memory, electrically programmable read only memory (“EPROM”), electrically erasable programmable read only memory (“EEPROM”)), any suitable media that is not fleeting or devoid of any semblance of permanence during transmission, and/or any suitable tangible media. As another example, transitory computer readable media can include signals on networks, in wires, conductors, optical fibers, circuits, or any suitable media that is fleeting and devoid of any semblance of permanence during transmission, and/or any suitable intangible media.

In a non-limiting example, subject interface 580 may be a display, such as a smart phone display, and the computer system 520 may be a smart phone. A subject may receive challenges 570 from a smart phone app, which may prompt a subject to engage in activity 590. Activity 590 may elicit an interaction with augmented environment 510. The immersive experience 550 may generate cognitive and emotional feedback via feedback 560 to the subject. In some configurations, the augmented environment may be composed of physical devices such as a smartphone, tablet, VR goggles, HoloLens, and the like.

In some configurations, real artifacts 540 include real objects, such as a bedroom, an office, a park, a city street, a desk, a puzzle, and the like. Synthetic artifacts 530 may include computer generated objects, such as artificial versions of the real artifacts mentioned, and may be included with the real objects to form the augmented environment 510. In a non-limiting example, the synthetic artifacts may include a puzzle configured to aid a subject in improving mental health. In another non-limiting example, the synthetic and real artifacts may be used in combination with an activity that uses the synthetic and real artifacts, which may be designed to improve mental health for the subject.

The augmented environment 510 may provide for a subject to be immersed within a virtual or mixed-reality experience that enhances medical treatments. Immersive experiences may be provided by virtual reality (VR) systems, augmented reality (AR), and mixed reality XR. In some configurations, the computer system 520 includes a processor, microprocessor, controller, or a combination of processors, microprocessor, and/or controllers that performs instructions stored in a memory to manipulate data stored in the memory to generate the augmented environment 510. The processor may be con figured with instructions to configure the processor to perform processes in accordance with the present disclosure. Immersive experiences may provide rewarding activities with little effort on behalf of a subject, and therefore a subject may require little motivation for participation in an immersive experience.

Subject interface 580 may include components for capturing data, such as (but not limited to) cameras, displays, and/or sensors, and the like. subject interface 580 may be configured to gather inputs and/or provide outputs.

In some configurations, the computer system 520 generating the immersive environment may also communicate with a healthcare analytic platform to receive a status of a subject or patient, and/or a treatment definition, which may be automatically selected by computer system 520 based upon complete health status, applied treatments, results of other patients having similar conditions to patient, and the like.

In some configurations, behavioral training programs may be used for negative symptoms that can include exposure to rewarding activities. Delivery of the behavioral training may be performed through immersive technology, in accordance with the present disclosure, which allows repeated controlled immersion in experiences which may be curated to enhance motivation towards and initial responsiveness to rewarding or positive experiences.

Mental health may be improved or promoted by exposing a subject to the augmented environment and deploying an activity designed to take the subject out of a patient mode, or negative mindset, and placing the subject into an adaptive mode. In a non-limiting example, the augmented environment may include a depiction of an outdoor space that may elicit a reaction in the subject, such as from agoraphobia. By adjusting the activity and the augmented environment, the subject may adapt to the outdoor space in the augmented environment and thereby mitigate the symptoms of agoraphobia. After iteration of the activity with successful symptom mitigation, the subject may be prepared for mitigated agoraphobia symptoms in the real world, or may no longer be afraid of the outdoors. Augmented reality may allow for transferrable progress, learning, or adaptability, and the like, from the augmented reality environment into the real world.

In some configurations, as the treatment protocol progresses and the activity is iterated, machine learning may be used to improve the performance of the system, or to automatically adjust the activity or progress the treatment protocol. In some configurations, as the method is used across multiple subjects and over time, data may be collected regarding the subjects, what produced successful outcomes, how the system performed, and the like, and the collected data may be used to train a machine learning routine.

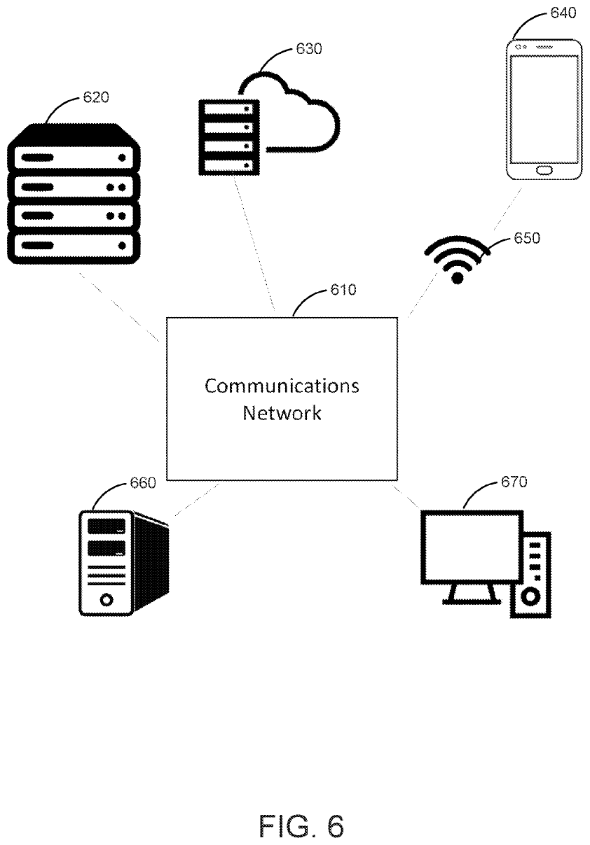

Referring to FIG. 6, a diagram of a non-limiting example system is shown capable of generating an immersive environment that provides immersive experience treatments. A communications network 610 may be configured to provide for intercommunication of devices, such as the internet in a non-limiting example. Servers 620 are connected to the communications network 610. Each of the servers 620 may be one or more interconnected servers that execute processes providing cloud services 630 to subjects over the network. Cloud services 630 may be one or more applications executed by one or more servers 620 to provide data and/or executable applications to devices over a network. The servers 620 may include any number of servers and any additional number of server systems 660 may be connected to the network to provide cloud services 630. Network interface may allow for immersive experiences to be transmitted to a subject and for receiving data over the network. Peripherals and/or network interfaces (not shown) can be used to gather inputs that can be used for a variety of purposes including, but not limited to, capture and/or analyze an individual's responses to an immersive experience.

In some configurations, an immersive environment system that uses systems and methods that provide immersive experience treatments may be provided by a process being executed on a server 620 and/or a group of server systems 660 communicating over a network. Treatment experiences can be generated at a server 620 and provided to a patient on a personal device 640. Data from individual use of the system can be collected and analyzed.

Subjects, such as patients, may use personal devices 640 that connect to the network to perform processes that provide immersive experience treatments. Subjects may also use computer device 670, such as a desktop computer, connected to network. Non-limiting examples of a personal device 640, or a computer device 670, include a laptop computer, a smart television, a game console, a smart phone or other device that connects to the network via a wired or wireless connection, such as wireless connection 650.

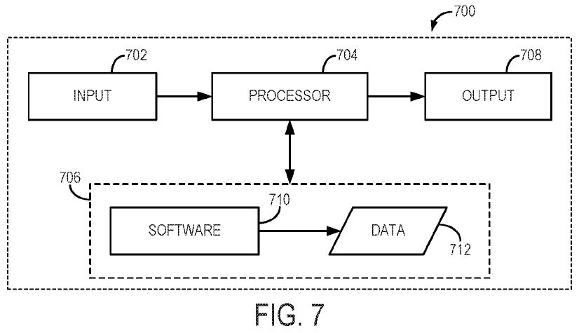

Referring to FIG. 7, a block diagram of an example of a computer system 700 that can perform the methods described in the present disclosure is shown. The computer system 700 generally includes an input 702, at least one hardware processor 704, a memory 706, and an output 708. Thus, the computer system 700 is generally implemented with a hardware processor 704 and a memory 706.

In some embodiments, the computer system 700 can be a server. The computer system 700 may also be implemented, in some examples, by a workstation, a notebook computer, a tablet device, a mobile device, a multimedia device, a network server, a mainframe, one or more controllers, one or more microcontrollers, a telehealth application, or any other general-purpose or application-specific computing device.

The computer system 700 may operate autonomously or semi-autonomously, or may read executable software instructions from the memory 706 or a computer-readable medium (e.g., a hard drive, a CD-ROM, flash memory), or may receive instructions via the input 702 from a user, or any another source logically connected to a computer or device, such as another networked computer or server. Thus, in some embodiments, the computer system 700 can also include any suitable device for reading computer-readable storage media.

In general, the computer system 700 is programmed or otherwise configured to implement the methods and algorithms described in the present disclosure. For instance, the computer system 700 can be programmed to implement an immersive technology to address mental illness or to promote mental health.

The input 702 may take any suitable shape or form, as desired, for operation of the computer system 700, including the ability for selecting, entering, or otherwise specifying parameters consistent with performing tasks, processing data, or operating the computer system 700. In some aspects, the input 702 may be configured to receive data, such as data acquired with a scoreboard. Such data may be processed as described above to generate an immersive experience. In addition, the input 702 may also be configured to receive any other data or information considered useful for assessing a subject's success using the methods described above.

Among the processing tasks for operating the computer system 700, the one or more hardware processors 704 may also be configured to carry out any number of post-processing steps on data received by way of the input 702. Post-processing may include updating the mental health scoreboard based on the subject's success.

The memory 706 may contain software 710 and data 712, such as data acquired with a scoreboard, and may be configured for storage and retrieval of processed information, instructions, and data to be processed by the one or more hardware processors 704. In some aspects, the software 710 may contain instructions directed to generate an immersive experience and to assess a subject's success.

In addition, the output 708 may take any shape or form, as desired, and may be configured for displaying a subject's results, in addition to other desired information.

The present disclosure has described one or more preferred embodiments, and it should be appreciated that many equivalents, alternatives, variations, and modifications, aside from those expressly stated, are possible and within the scope of the invention.

1. A computer-implemented method for improving mental health of a subject comprising:
a) obtaining, using a processor, a mental health treatment protocol based on an initial assessment of the subject;
b) generating, using the processor, an immersive experience for the subject to implement the mental health treatment protocol and to facilitate the subject to enter an adaptive mode;
c) generating, using the processor, a challenge activity for the subject in the immersive experience and assessing the subject's success with the challenge activity associated with the immersive experience; and
d) updating, using the processor, the mental health treatment protocol based upon the success of the subject in the immersive experience.

2. The computer-implemented method of claim 1, wherein the immersive experience is at least one of a virtual reality, an augmented reality, a synthetic environment, or a mixed reality.

3. The computer-implemented method of claim 2, wherein the immersive experience is an augmented reality and wherein the augmented reality includes real artifacts overlaid with synthetic artifacts generated for the augmented reality.

4. The computer-implemented method of claim 3, wherein the challenge activity includes a puzzle and wherein the puzzle is configured to facilitate the subject to enter the adaptive mode.

5. The computer-implemented method of claim 1, wherein the challenge activity is configured to improve mental health of the subject.

6. The computer-implemented method of claim 5, wherein assessing the subject's success with the challenge activity includes determining at least one of a duration of the challenge activity, a difficulty of completing the challenge activity, retained learning from the challenge activity, or subject fatigue.

7. The computer-implemented method of claim 1, wherein the treatment protocol includes a plurality of treatment episodes, each of which include a plurality of generated immersive experiences.

8. The computer-implemented method of claim 7, wherein assessing the subject's success with the challenge activity is performed after each immersive experience, and subsequent immersive experiences are adjusted based upon a preceding assessment of the subject's success with the challenge activity.

9. The computer-implemented method of claim 1, wherein assessing the subject's success with the challenge activity includes using at least one of a microphone, a video camera, and a biometric sensor to acquire data of the performance of the subject.

10. The computer-implemented method of claim 1, wherein the initial assessment includes an assessment of at least one of avolition, asociality, or anhedonia in the subject.

11. The computer-implemented method of claim 1, further comprising calibrating a mental health scoreboard based on the initial assessment of the subject, and
wherein the mental health scoreboard includes scoring the subject in areas of at least one of experience, belief system, or functional targets.

12. A non-transitory computer-readable medium storing instructions that, when executed by a computer system, cause the computer system to perform a method comprising:
a) obtaining, using a processor, a mental health treatment protocol based on an initial assessment of the subject;
b) generating, using the processor, an immersive experience for the subject to implement the mental health treatment protocol and to facilitate the subject to enter an adaptive mode;
c) generating, using the processor, a challenge activity for the subject in the immersive experience and assessing the subject's success with the challenge activity associated with the immersive experience; and
d) updating, using the processor, the mental health treatment protocol based upon the success of the subject in the immersive experience.

13. The computer-readable medium of claim 12, wherein the immersive experience is at least one of a virtual reality, an augmented reality, a synthetic environment, or a mixed reality.

14. The computer-readable medium of claim 13, wherein the immersive experience is an augmented reality and wherein the augmented reality includes real artifacts overlaid with synthetic artifacts generated for the augmented reality.

15. The computer-readable medium of claim 14, wherein the challenge activity includes a puzzle and wherein the puzzle is configured to facilitate the subject to enter the adaptive mode.

16. The computer-readable medium of claim 12, wherein the challenge activity is configured to improve mental health of the subject.

17. The computer-readable medium of claim 16, wherein assessing the subject's success with the challenge activity includes determining at least one of a duration of the challenge activity, a difficulty of completing the challenge activity, retained learning from the challenge activity, or subject fatigue.

18. The computer-readable medium of claim 12, wherein the treatment protocol includes a plurality of treatment episodes, each of which include a plurality of generated immersive experiences.

19. The computer-readable medium of claim 18, wherein assessing the subject's success with the challenge activity is performed after each immersive experience, and subsequent immersive experiences are adjusted based upon a preceding assessment the subject's success with the challenge activity.

20. The computer-readable medium of claim 12, wherein assessing the subject's success with the challenge activity includes using at least one of a microphone, a video camera, and a biometric sensor to acquire data of the performance of the subject.

21. The computer-readable medium of claim 12, wherein the initial assessment includes an assessment of at least one of avolition, asociality, or anhedonia in the subject.

22. The computer-readable medium of claim 12, wherein the computer system is further configured to calibrate a mental health scoreboard based on the initial assessment of the subject, and
wherein the mental health scoreboard includes scoring the subject in areas of at least one of experience, belief system, or functional targets
